(19) United States
(12) Patent Application Publication
(10) Pub. No.: US 2006/0047247 A1
(43) Pub. Date: Mar. 2, 2006

(54) Catheter and methods of use and manufacture

(75) Inventor: Robert Anders, Scottsdale, AZ (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(73) Assignee: 3FI Products LLC

(21) Appl. No.: 10/924,697

(22) Filed: Aug. 24, 2004

(51) Int. Cl. A61M 5/178

(52) U.S. Cl. 604/164.08; 604/288

(57) ABSTRACT

In an embodiment, a catheter apparatus includes a catheter and a substantially soluble insertion point extending beyond a distal end of the catheter. The substantially soluble insertion point may be connected to a substantially soluble needle shaft or to a non-soluble needle shaft. The catheter apparatus includes a partial retraction mechanism, in an embodiment, which enables the substantially soluble insertion point to be retracted to a position within the catheter. In other embodiments, the substantially soluble insertion point is fully retractable (i.e., removable) or is non-retractable.

### TECHNICAL FIELD

This invention relates generally to catheters and, in particular, to an intravenous catheter in which at least the insertion point is substantially soluble, and methods of making and using embodiments of a catheter.

### BACKGROUND

Intravenous (IV) catheters may be used to administer fluids directly into a patient's vascular system. An IV catheter includes a flexible tube, which is attached at a proximal end to a catheter connector. A handheld placement device, which includes a sharp tip needle, is used to insert the IV catheter into a patient's vein. For an “over the needle” type of catheter, prior to insertion, a needle is positioned within the catheter so that the needle's tip extends slightly beyond the distal end of the catheter. The opposite end of the needle extends through the catheter connector and is connected to a needle hub.

To insert the catheter, a person (e.g., a health care worker) inserts the needle point through the patient's skin and into the patient's vein. Via the catheter connector, the person pushes the distal end of the catheter toward and beyond the needle point, thus locating the catheter's distal end within the vein. Once the catheter is positioned in this way, the person withdraws the needle by applying pressure to the patient's vein near the insertion site, and grasping and pulling the needle hub in a direction away from the insertion site. This removes the needle and needle hub from the catheter and catheter connector. An exposed portion of the catheter is taped to the patient's skin, and the catheter connector is attached to a source of fluid. The fluid then flows through the catheter into the patient's vein.

After a catheter needle has been used, it may include various, dangerous, blood-borne pathogens. Accordingly, a used catheter insertion needle is considered a contaminated bio-hazard. An inadvertent “stick” from a used needle may result in the exposed person contracting hepatitis, AIDS, or some other communicable disease. Therefore, strict regulations for the use and disposal of used needles exist, in order to reduce the likelihood that health care workers and others may inadvertently expose themselves to blood and other bodily substances that may be present on or within used needles.

However, even with strict regulations and extensive education, inadvertent needle sticks continue to occur at alarming rates. Needle sticks have been experienced by persons administering shots, drawing blood, inserting catheters, and performing other actions with needles. Further, an improperly disposed of, used needle may stick other persons long after the initial use of the needle has occurred. Many of these inadvertent needle sticks have exposed unintended victims to deadly diseases, resulting in a significant amount of deaths.

### BRIEF DESCRIPTION OF THE DRAWINGS

FIG. 1 illustrates a perspective view of a catheter and catheter insertion device, in accordance with an embodiment of the inventive subject matter;

FIG. 2 illustrates a cross-sectional view of a catheter and catheter insertion device, in accordance with an embodiment;

FIG. 3 illustrates a cross-sectional view of a catheter and catheter insertion device, in accordance with another embodiment;

FIG. 4 illustrates an enlarged, cross sectional view of a catheter insertion device retraction mechanism in a non-retracted position, in accordance with an embodiment;

FIG. 5 illustrates an enlarged, cross sectional view of the retraction mechanism of FIG. 4 in a retracted position, in accordance with an embodiment;

FIG. 6 illustrates a cross-sectional view of a catheter and a catheter insertion device in a retracted position, in accordance with an embodiment;

FIG. 7 illustrates a perspective view of a first portion of a retraction mechanism, in accordance with an embodiment;

FIG. 8 illustrates a cross-sectional view of a second portion of a retraction mechanism, in accordance with an embodiment;

FIG. 9 illustrates a cross-sectional view of the second portion of the retraction mechanism of FIG. 8 along section lines 9-9, in accordance with an embodiment;

FIG. 10 illustrates a cross-sectional view of the second portion of the retraction mechanism of FIG. 8 along section lines 9-9, in accordance with another embodiment;

FIG. 11 illustrates a flowchart of a method for using a catheter with a retractable, soluble insertion point, in accordance with an embodiment;

FIG. 12 illustrates a cross-sectional view of a catheter shortly after insertion of the soluble insertion point into a vein, in accordance with an embodiment;

FIG. 13 illustrates a cross-sectional view of the catheter of FIG. 12 after retraction of the soluble insertion point into the distal catheter end, in accordance with an embodiment;

FIG. 14 illustrates a cross-sectional view of the catheter of FIG. 13 after further advancement of the catheter into a patient's vein, in accordance with an embodiment;

FIG. 15 illustrates a cross-sectional view of the catheter of FIG. 14 after a period of time has elapsed during which the soluble insertion point has partially dissolved;

FIG. 16 illustrates a cross-sectional view of the catheter of FIG. 15 after a second period of time has elapsed during which the soluble insertion point and needle have fully dissolved;

FIG. 17 illustrates a flowchart of a method for manufacturing a catheter having a soluble insertion point, in accordance with an embodiment;

FIG. 18 illustrates a flowchart of a method for manufacturing a catheter having a soluble insertion point, in accordance with another embodiment; and

FIG. 19 illustrates a flowchart of a method for fabricating a soluble insertion point or needle, in accordance with an embodiment.

### DESCRIPTION OF THE EMBODIMENTS

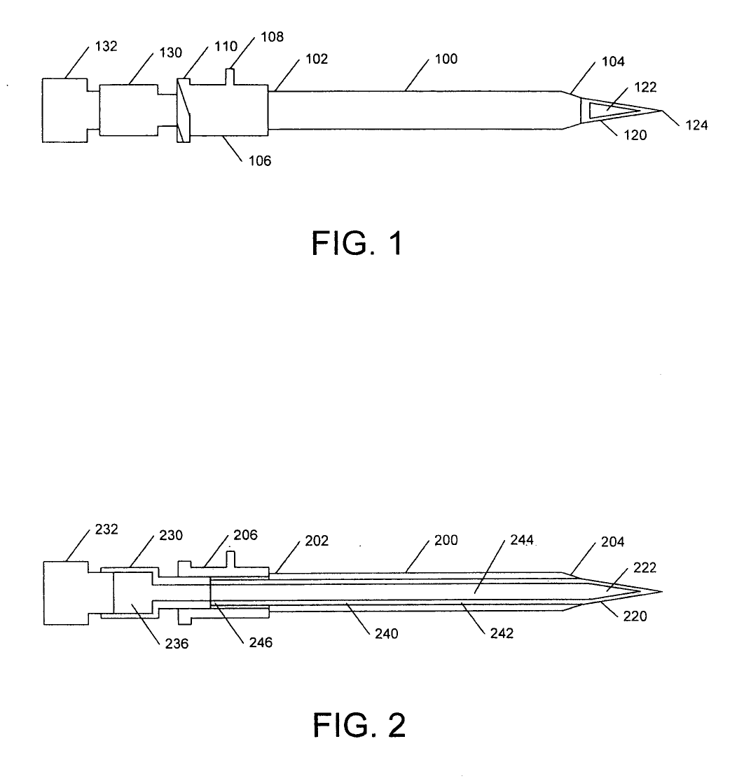

FIG. 1 illustrates a perspective view of a catheter 100 and catheter insertion device, in accordance with an embodiment of the inventive subject matter. Catheter 100 may be, for example, an intravenous (IV) catheter, which is used to deliver fluids to a patient's vascular system. In other embodiments, catheter 100 may be intended for other purposes, such as, for example, a catheter used to drain fluids from a patient's body or to provide access to an area of the body (e.g., the chest cavity).

Catheter 100 includes a hollow catheter interior (not depicted in FIG. 1), a proximal catheter end 102, and a distal catheter end 104. The hollow catheter interior extends between proximal catheter end 102 and distal catheter end 104, providing for the flow of fluids between and through openings at the two ends. In an embodiment, proximal catheter end 102 is coupled to a catheter connector 106. In an embodiment, catheter connector 106 includes an extension 108 or ridge, which facilitates advancement of the catheter tube in relation to a catheter insertion needle. In addition, in an embodiment, catheter connector 106 includes a connector portion 110 (e.g., a threaded portion), which provides for attachment of the catheter to fluid-delivery tubing after the catheter 100 has been inserted at the intended location (e.g., into a patient's vein).

The catheter insertion device includes an insertion point 120, formed from a hardened, substantially soluble material. The term “soluble,” as used herein, means substantially soluble, and the term “soluble material” means a material that includes one or more substantially soluble substances. In an embodiment, the soluble material dissolves in a liquid solution, which may or may not include a significant amount of water as a component. In another embodiment, the soluble material dissolves in an aqueous solution (i.e., a liquid solution having a significant amount of water as a component). The term “substantially,” as used with “soluble” herein, encompasses complete or partial dissolution. The term “soluble insertion point” is defined to mean that a portion of the insertion point is soluble in a liquid so that the point will be blunted to the extent that it no longer will pierce skin under normal conditions of use.

Prior to initial use of the catheter 100 and the catheter insertion device, soluble insertion point 120 extends beyond the distal catheter end 104. In an embodiment, soluble insertion point 120 includes an internal channel (not depicted in FIG. 1), which has an opening 122 in proximity to a sharp end 124. The internal channel extends from opening 122 in a direction coaxial with the hollow catheter interior.

In an embodiment, soluble insertion point 120 forms a distal end portion of a soluble needle, which includes a shaft that also may be substantially formed from a substantially soluble material. In an alternate embodiment, the shaft of the needle is formed from stainless steel or another non-soluble material. The needle is attached at a proximal needle end to a substantially hollow needle hub 130. Prior to use, an end cap 132 may substantially seal an interior blood collection chamber (not depicted in FIG. 1) of the needle hub 130.

FIG. 2 illustrates a cross-sectional view of a catheter 200 and catheter insertion device, in accordance with an embodiment. Catheter 200 includes a hollow catheter interior, a proximal catheter end 202, and a distal catheter end 204. In an embodiment, proximal catheter end 202 is coupled to a catheter connector 206.

The catheter insertion device includes a needle 240, which includes a substantially soluble point 220. In an embodiment, soluble point 220 is integrally connected to a shaft 242 of needle 240. In an alternate embodiment, soluble point 220 is otherwise attached to shaft 242. A hollow needle channel 244 extends from a proximal needle end 246 to and through opening 222 of soluble point 220 in a direction coaxial to the hollow catheter interior.

Soluble point 220 and shaft 242 may be formed from the same material or different materials. In an embodiment, soluble point 220 and shaft 242 are substantially formed from one or more bio-compatible materials. In other embodiments, soluble point 220 and shaft 242 may be formed from other types of soluble materials. In still another embodiment, shaft 242 is formed from stainless steel or another non-soluble material. As discussed previously, prior to initial use of the catheter 200 and the catheter insertion device, soluble insertion point 220 extends beyond the distal catheter end 204.

Proximal needle end 246 is attached to a substantially hollow needle hub 230. Prior to use, an end cap 232 may substantially seal an interior blood collection chamber 236 of the needle hub 230. During use, if the soluble point 220 is properly inserted into a vein, a small amount of blood may flow through the catheter needle and into the blood collection chamber 236. In an embodiment, the blood collection chamber 236 is formed from a material that enables the catheter user to observe blood in the blood collection chamber 236.

In an embodiment, after the catheter 200 has been properly inserted (e.g., into a vein), the soluble insertion point 220 may be removed from the catheter tube by pulling the needle hub 230 and the needle 240 in a direction away from the insertion site. The needle 240 and hub 230 may then be disposed of in a suitable disposal container. In an embodiment, the disposal container includes a fluid within which the soluble point may dissolve. In an alternate embodiment, the soluble insertion point 220 may remain in the vein until it dissolves in the blood or other fluids. In such an embodiment, the needle may not need to be retracted and, accordingly, the needle hub and the catheter connector may be integrally connected or otherwise attached together.

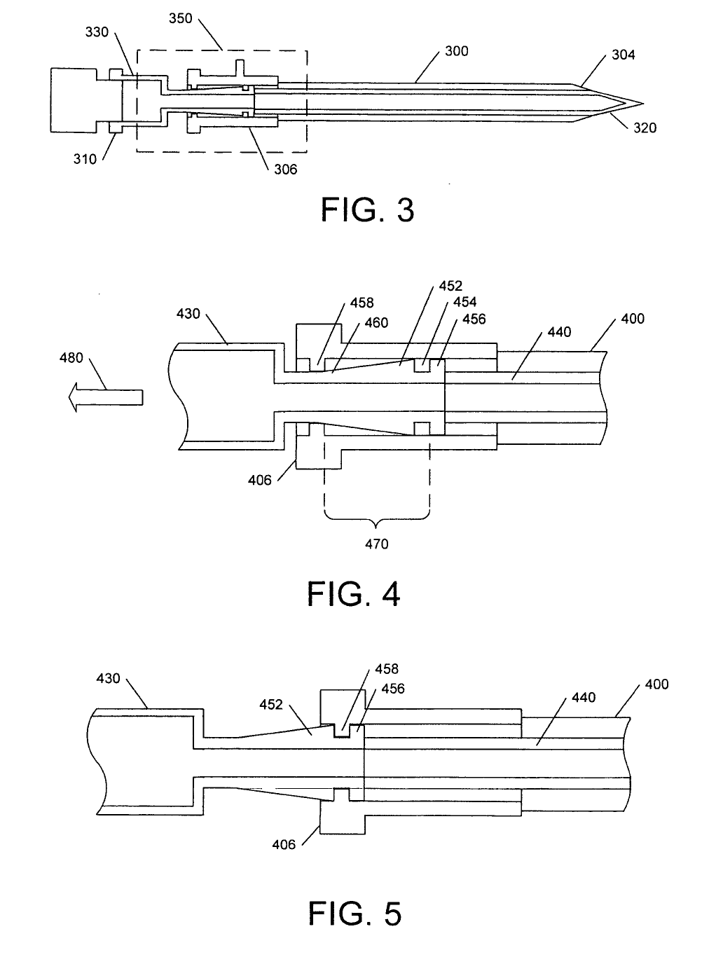

FIG. 3 illustrates a cross-sectional view of a catheter 300 and catheter insertion device, in accordance with another embodiment. Catheter 300 and the catheter insertion device depicted in FIG. 3 may be substantially similar to the catheter 200 and catheter insertion device depicted in FIG. 2, except that the catheter insertion device of FIG. 3 may additionally include a partial needle retraction mechanism. In an embodiment, the partial needle retraction mechanism is located in an area 350 proximate to catheter connector 306 and needle hub 330. In addition, in an embodiment, needle hub 330 includes a connector portion 310 (e.g., a threaded portion), which provides for attachment of the needle hub to fluid-delivery tubing after the catheter 300 has been inserted at the intended location (e.g., into a patient's vein).

The catheter 300 and catheter insertion device depicted in FIG. 3 are in a non-retracted position. In such a position, insertion point 320 extends outside of and beyond distal catheter end 304.

FIG. 4 illustrates an enlarged, cross sectional view of an area (e.g., area 350) of a catheter insertion device retraction mechanism in a non-retracted position, in accordance with an embodiment. In an embodiment, the retraction mechanism includes complementary mechanical mechanisms associated with catheter connector 406 and needle hub 430. In an embodiment, needle hub 430 includes one or more angled glides 452, one or more notches 454, and one or more first glide stop mechanisms 456. Catheter connector 406 includes one or more second glide stop mechanisms 458, which may be shaped to be engagable with notches 454. In an alternate embodiment, catheter connector 406 includes one or more glides, notches, and first glide stop mechanisms, and needle hub 430 includes one or more second glide stop mechanisms. In other words, in an alternate embodiment, portions of a partial retraction mechanism may be located on opposite ones of catheter connector 406 and needle hub 430 than is illustrated in FIG. 4.

In a non-retracted position, first and second glide stop mechanisms 456, 458 are respectively positioned so that second glide stop mechanisms 458 are proximate to a first section 460 of angled glides 452, where the first section 460 is located approximately a retraction distance 470 from notches 454 and/or first glide stop mechanisms 456.

To initiate partial retraction of a needle 440 attached to needle hub 430, a person may pull needle hub 430 in a direction indicated by arrow 480. The pulling force causes notches 454 to move in a direction toward second glide stop mechanisms 458, while second glide stop mechanisms 458 slideably engage angled glides 452. Eventually, notches 454 will reach second glide stop mechanisms 458, and second glide stop mechanisms 458 will engage with notches 454, placing the retraction mechanism in a retracted position. Further movement of second glide stop mechanisms 458 with respect to notches 454 is limited by substantially parallel sides of angled glides 452 and first glide stop mechanisms 456.

FIG. 5 illustrates an enlarged, cross sectional view of the retraction mechanism of FIG. 4 in a retracted position, in accordance with an embodiment. As illustrated in FIG. 5, second glide stop mechanisms 458 are engaged with notches of the needle hub 430 (i.e., notches 454, FIG. 4). Further movement of second glide stop mechanisms 458 with respect to the notches is limited by substantially parallel sides of angled glides 452 and first glide stop mechanisms 456. The at least partial retraction of needle 440 with respect to catheter connector 406 also results in the at least partial retraction of the insertion point (e.g., point 320, FIG. 3) with respect to the distal end of the catheter 400 (e.g., distal end 304, FIG. 3).

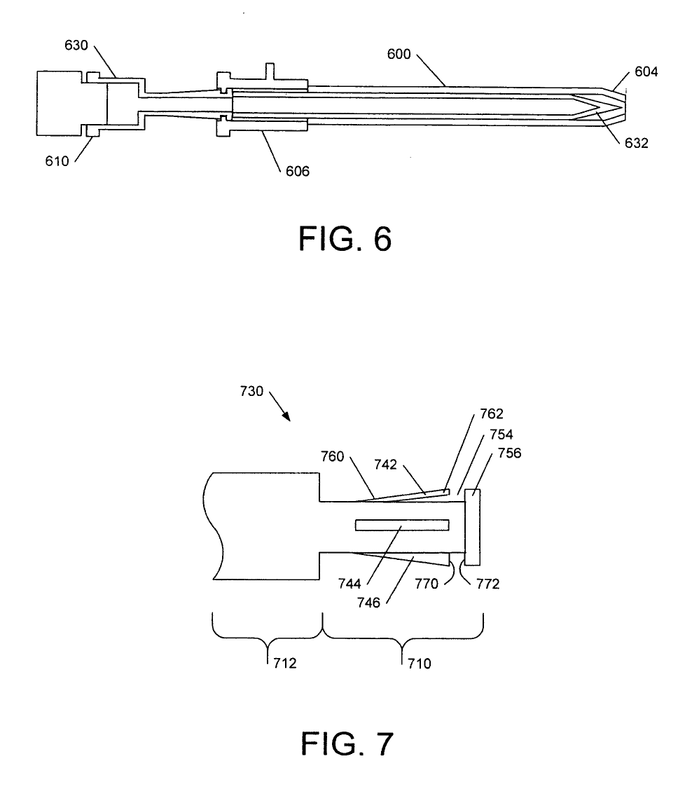

FIG. 6 illustrates a cross-sectional view of a catheter and a catheter insertion device in a retracted position, in accordance with an embodiment. In the retracted position, second glide stop mechanisms (e.g., mechanisms 458, FIG. 5) are held in place with respect to notches by substantially parallel sides of angled glides (e.g., glides 452, FIG. 5) and first glide stop mechanisms (e.g., mechanisms 456, FIG. 5).

In this position, insertion point 632 is retracted past distal catheter end 604 to a position within catheter 600. Although insertion point 632 is illustrated to be retracted to a position just inside distal catheter end 604, in other embodiments, the retraction distance may be longer, thus resulting in insertion point 632 being retractable to a position further inside catheter 600.

In an embodiment, needle hub 630 includes a connector portion 610 (e.g., a threaded portion), which provides for attachment of the catheter to fluid-delivery tubing after the catheter 600 has been inserted at the intended location (e.g., into a patient's vein). In an alternate embodiment, needle hub 630 may be configured so that fluid-delivery tubing may fit over needle hub 630 and connect instead to the catheter connector 606.

In an embodiment, the needle retraction mechanism includes at least two portions, where a first portion is connected to the needle hub, and the second portion is connected to the catheter connector. FIG. 7 illustrates a perspective view of a first portion of a retraction mechanism, in accordance with an embodiment. The first retraction mechanism portion is integrally attached to needle hub 730, in an embodiment. In an alternate embodiment, the first portion is otherwise connected to the needle hub. Needle hub 730 generally includes a shaft section 710 and a blood collection chamber portion 712 (only part of the blood collection chamber portion is illustrated in FIG. 7).

On the shaft section 710 of needle hub 730, the first retraction mechanism portion includes one or more angled glides 742, 744, 746, one or more slots 754, and one or more retraction stop mechanisms 756, in an embodiment. Although three angled glides are illustrated in FIG. 7, the first retraction mechanism may include as few as one angled glide or several (e.g., four or more) angled glides distributed around the circumference of the shaft 710.

In an embodiment, one or more angled glides (e.g., glide 742) may include an elongated member, which is attached to shaft 710 at a first end 760, and free at a second end 762. A space may exist between the second end 762 and an adjacent portion of the shaft 710, allowing for deflection of the second end 762 toward shaft 710 as the catheter connector's retraction mechanism (e.g., mechanism 458, FIG. 4) slides along the elongated member during needle retraction. In an embodiment, the elongated member has a sufficient spring coefficient to enable it to deflect toward shaft 710, and then snap back toward its original, undeflected position, after the catheter connector's retraction mechanism has slid into slot 754.

In another embodiment, one or more angled glides (e.g., glide 746) may include a wedge-shaped member, which is in contact with shaft 710 along a substantial portion of the glide's length. Once the catheter connector's retraction mechanism has slid into slot 754, it is held in place by substantially parallel sides 770, 772 of angled glides 742, 744, 746 and retraction stop mechanism 756.

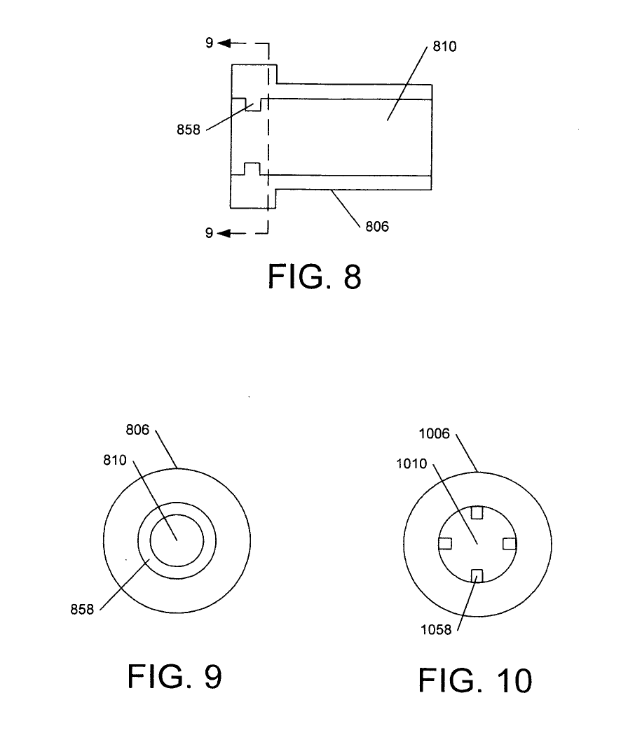

FIG. 8 illustrates a cross-sectional view of a second portion of a retraction mechanism, in accordance with an embodiment. The second retraction mechanism portion is integrally attached to catheter connector 806, in an embodiment. In an alternate embodiment, the second portion is otherwise connected to the catheter connector.

The second retraction mechanism portion includes one or more retraction stop mechanisms 858, in an embodiment, which extend into an interior channel 810 of catheter connector 806. As described previously, retraction stop mechanisms 858 slideably engage one or more of a needle hub's angled glides (e.g., glides 742, 744, 746), and engage one or more needle hub slots (e.g., slot 754, FIG. 7). In an embodiment, retraction stop mechanism 858 includes a ring, as will be illustrated and described in conjunction with FIG. 9. In another embodiment, retraction stop mechanism 858 includes one or more tabs, as will be described in conjunction with FIG. 10.

FIG. 9 illustrates a cross-sectional view of the second portion of a retraction mechanism of FIG. 8 along section lines 9-9, in accordance with an embodiment. The second portion of the retraction mechanism includes a ring 858, which extend inward into the interior channel 810 of catheter hub 806.

FIG. 10 illustrates a cross-sectional view of the second portion of a retraction mechanism of FIG. 8 along section lines 9-9, in accordance with another embodiment. The second portion of the retraction mechanism includes one or more tabs 1058, which extend inward into the interior channel 1010 of catheter hub 1006.

Besides using a retraction mechanism having substantially a same design as the retraction mechanisms illustrated in conjunction with FIGS. 3-10, other types of retraction mechanisms alternatively could be used, as may be apparent to those of skill in the art based on the description herein. Numerous types of retraction mechanisms may be used to achieve the same purpose of retracting the insertion point into the catheter.

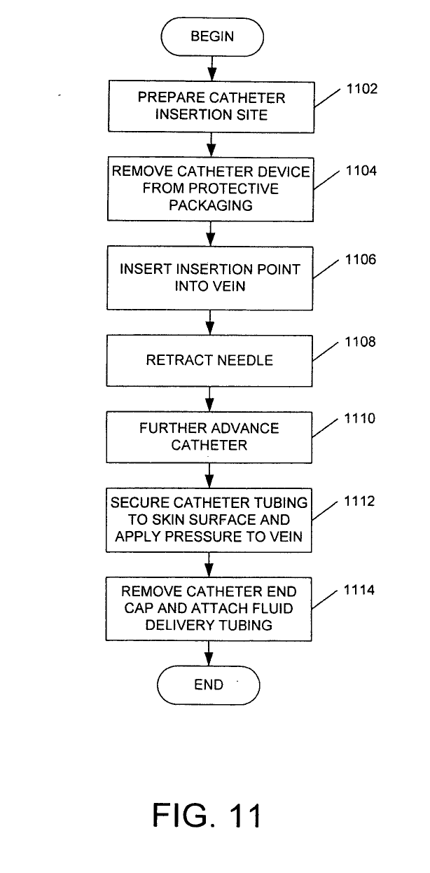

FIG. 11 illustrates a flowchart of a method for using a catheter with a partially-retractable, substantially soluble insertion point, in accordance with an embodiment. The method begins, in block 1102, by preparing a catheter insertion site on a body. This may include, for example, a person visually locating a candidate vein by inspection of the surface of a patient's skin, and cleaning the skin overlying the vein with an antibacterial solution or disinfectant (e.g., iodine and/or alcohol).

In block 1104, the catheter device (i.e., the assembled catheter, catheter hub, needle, needle hub, and end cap) is removed from protective packaging. In an embodiment, the packaging is used to ensure that the catheter device remains sterile prior to use.

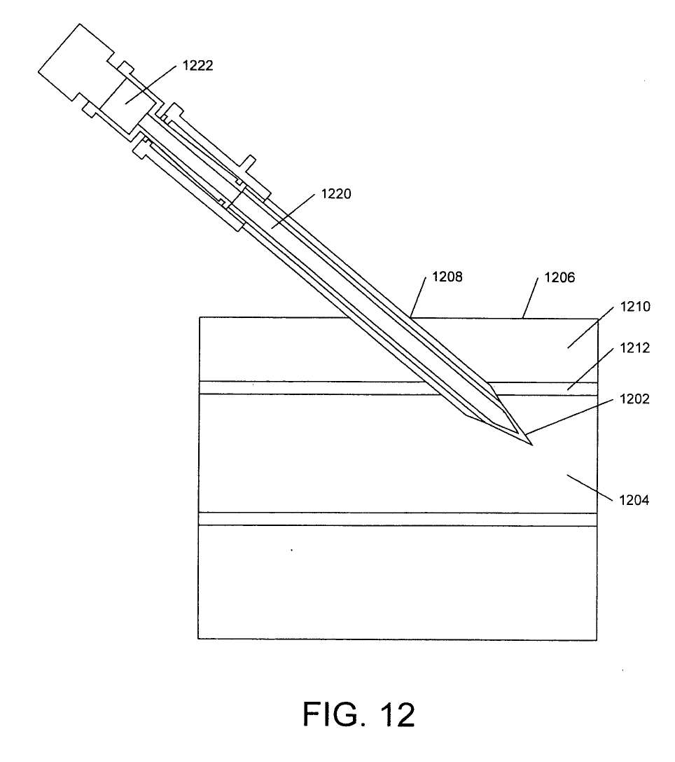

In block 1106, the insertion point is inserted at the insertion site through the surface of the skin and into the patient's vein. FIG. 12 illustrates a cross-sectional view of a catheter shortly after insertion of the soluble insertion point 1202 into an interior channel of a vein 1204, in accordance with an embodiment. The insertion point 1202 is inserted through the surface 1206 of the patient's skin at an insertion site 1208. The insertion point 1202 is then advanced through any intermediate tissue 1210, through the vein's top lumen 1212, and into the interior channel 1204. When so inserted, blood located in the interior channel 1204 may flow up through the needle channel 1220 and into blood collection chamber 1222. Proper insertion into a vein may be verified by observing the blood in the blood collection chamber 1222.

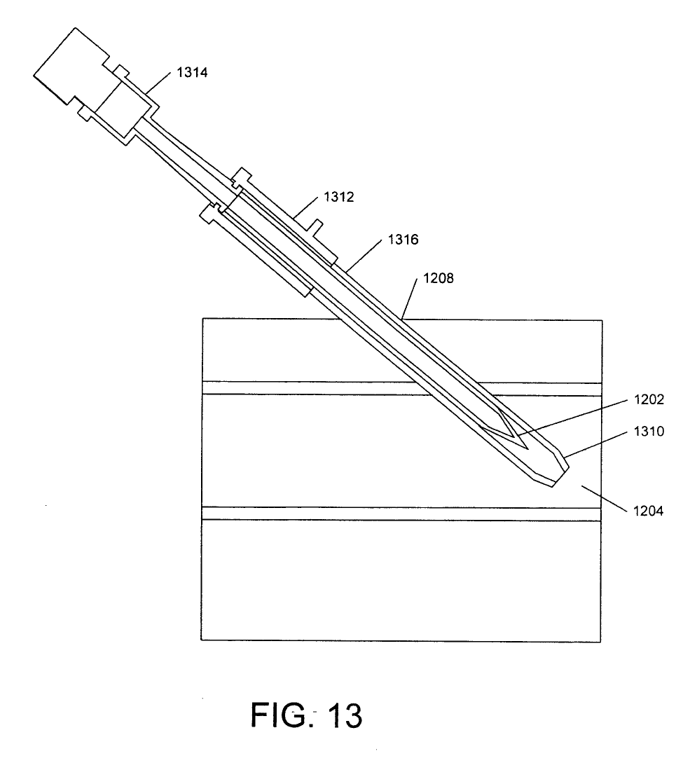

Referring back to FIG. 11, the needle is retracted in block 1108. In an embodiment, the needle is partially retracted, as facilitated by the use of a partial retraction mechanism. FIG. 13 illustrates a cross-sectional view of the catheter of FIG. 12 after retraction of the soluble insertion point 1202 into the distal catheter end 1310, in accordance with an embodiment. As described previously, the needle may be partially retracted by holding the catheter connector 1312 stationary with respect to the insertion site 1208, and pulling the needle hub 1314 outwardly away from the body. Alternatively, the needle hub 1314 may be held stationary with respect to the insertion site 1208, and the catheter connector 1312 may be pushed toward the insertion site 1208, thus advancing the distal catheter end 1310 beyond the insertion point 1202 and further into the interior channel 1204 of the vein. Either way, once partially retracted, the insertion point 1202 is positioned inside the catheter tube 1316.

In another embodiment, the needle and needle hub may be fully retracted (i.e., completely removed from the catheter and catheter connector) and discarded in an appropriate waste container. In an embodiment, a fully retractable needle (e.g., as illustrated in FIG. 2) may be placed into a waste container that contains a solution that substantially or completely dissolves the soluble material of the insertion point and/or the entire needle. Accordingly, the sharp insertion point may eventually substantially dissolve, thus eliminating the hazardous medical sharp. FIGS. 11-16 illustrate embodiments having a partially retractable needle. However, it is to be understood that embodiments of the inventive subject matter also may apply to a fully retractable catheter device.

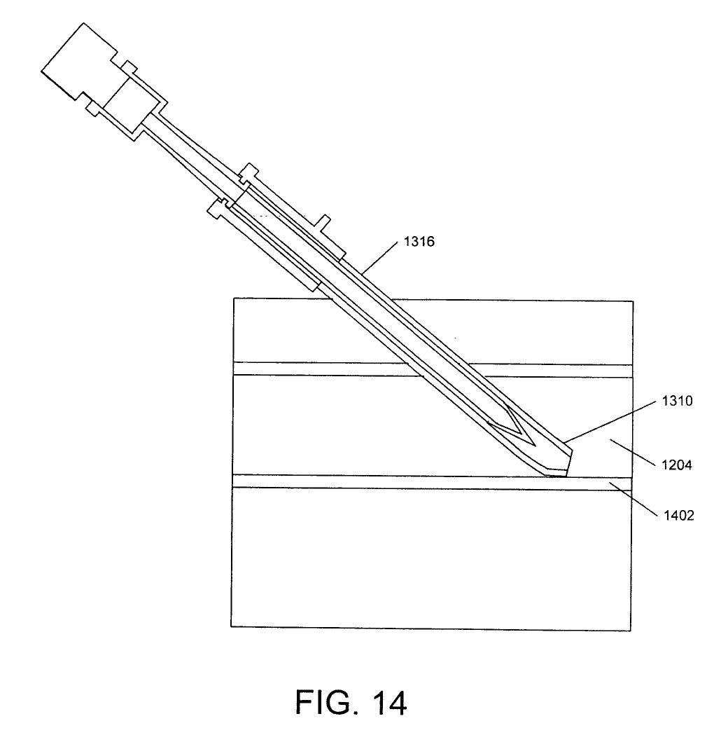

Referring again to FIG. 11, in block 1110, the distal catheter end is advanced further into the patient's vein. FIG. 14 illustrates a cross-sectional view of the catheter of FIG. 13 after further advancement of the catheter into a patient's vein, in accordance with an embodiment. During advancement, the distal catheter end 1310 may contact a bottom side of the vein's lumen 1402. However, for normal veins, the distal catheter end 1310 will lack sufficient sharpness to puncture the lumen 1402, in an embodiment. Accordingly, the catheter tube 1316 may bend while being advanced further into the interior channel of the vein 1204.

Referring again to FIG. 11, in block 1112, the catheter connector and/or tubing are secured to the surface of the patient's skin using medical tape, for example, and pressure is applied to the patient's vein in proximity to the insertion site, in order to stem the flow of blood through the vein. The catheter end cap is removed, in block 1114, and fluid-delivery tubing is attached to the device. In an embodiment, the needle hub is designed to attach to the fluid-delivery tubing connector. In another embodiment, the tubing connector fits over the needle hub and is attachable to the catheter connector.

As described previously, the insertion point is formed from a substantially soluble material, and may substantially dissolve in physiological fluids of a mammal (e.g., blood, urine, lymphatic fluid, gastro-intestinal fluid, saliva, and the like) and/or other fluids (e.g., the fluid being administered to the patient). In addition, in an embodiment, the needle shaft also is formed from a substantially soluble material. Accordingly, over a period of time, the insertion point and/or the needle may substantially or completely dissolve.

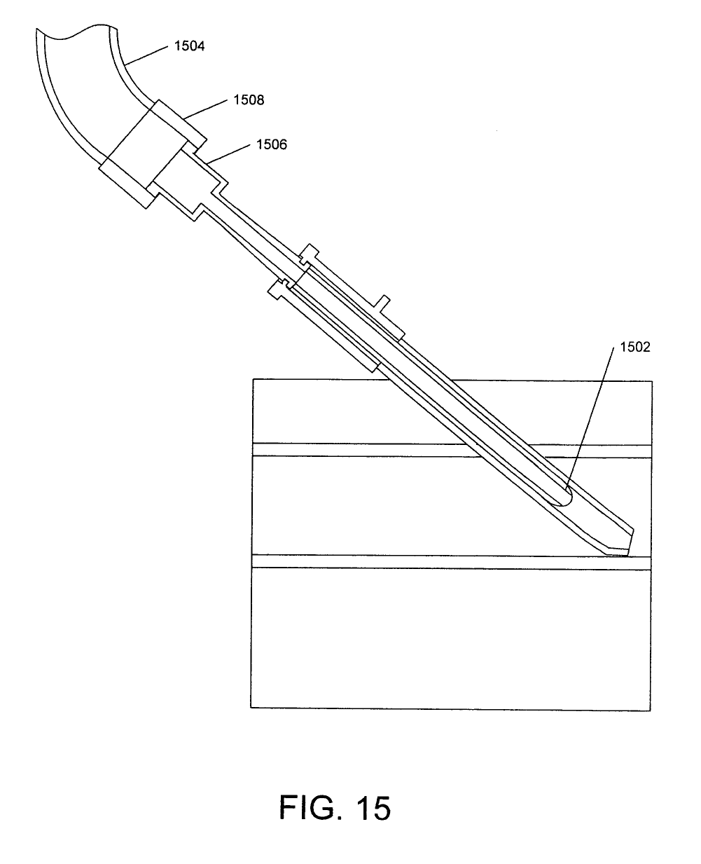

FIG. 15 illustrates a cross-sectional view of the catheter of FIG. 14 after a period of time has elapsed during which the soluble insertion point 1502 has partially dissolved. FIG. 15 also illustrates fluid-delivery tubing 1504 attached to needle hub 1506 via a tubing connector 1508.

Insertion point 1502 may obtain a blunted shape when it is partially dissolved. In an embodiment, insertion point 1502 is formed from a material that dissolves within a relatively short amount of time (e.g., minutes). In an alternate embodiment, insertion point 1502 may take a longer amount of time to dissolve (e.g., hours or days). Eventually, in an embodiment, insertion point 1502 and the needle shaft (if it is formed from a soluble material) will be substantially or completely dissolved.

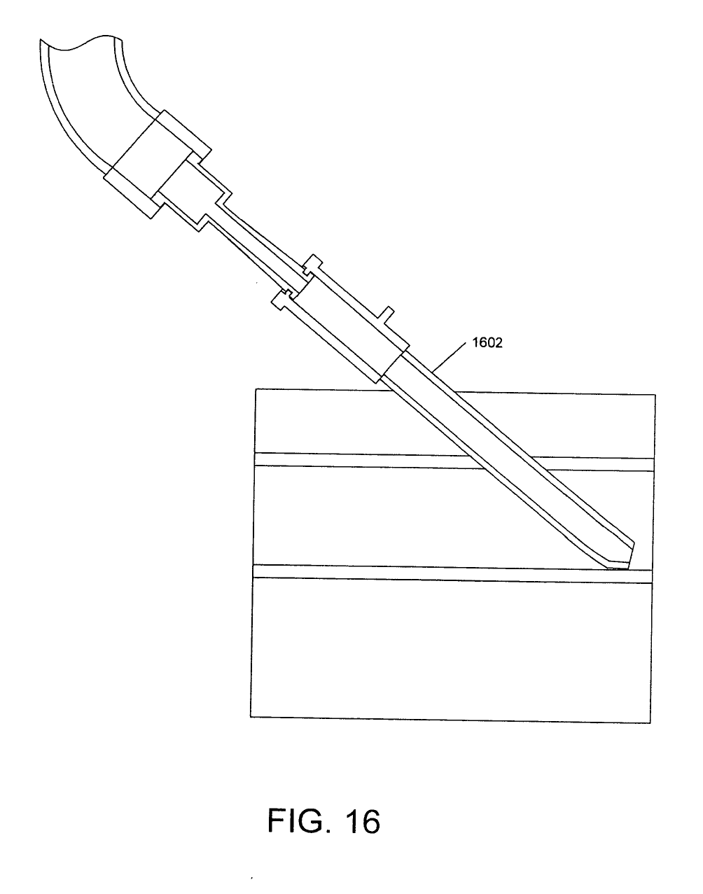

FIG. 16 illustrates a cross-sectional view of the catheter of FIG. 15 after a second period of time has elapsed during which the soluble insertion point and needle have fully dissolved. With the soluble insertion point and needle fully dissolved, only the catheter tube 1602 remains in the patient's vein. In an alternate embodiment, a substantially soluble insertion point and/or needle may have one or more reinforcing structures or materials, which may or may not completely dissolve. Accordingly, after substantial dissolution of the soluble material, the one or more reinforcing structures or materials may remain.

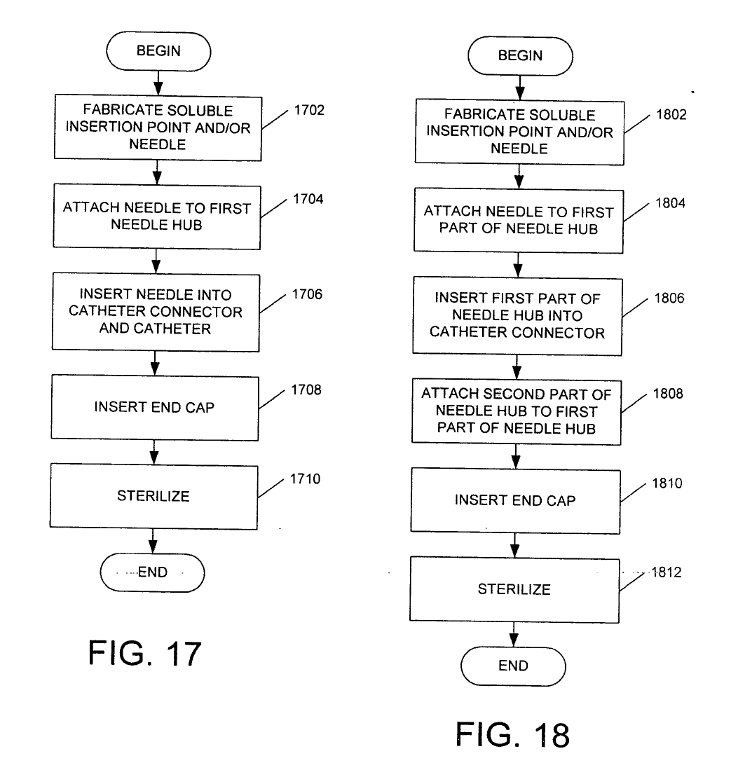

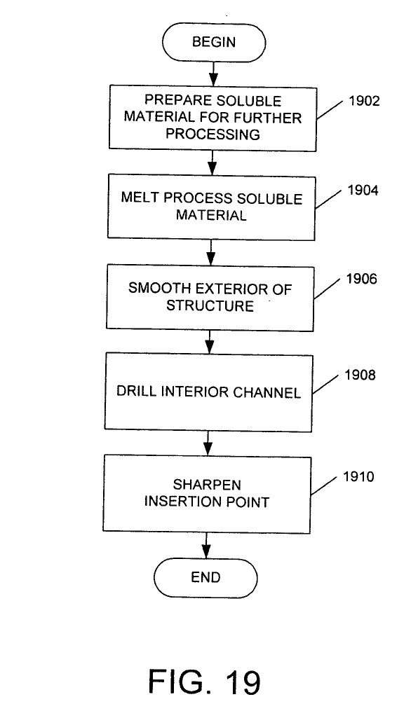

FIG. 17 illustrates a flowchart of a method for manufacturing a catheter having a substantially soluble insertion needle, in accordance with an embodiment. The method begins, in block 1702, by fabricating an insertion point. In an embodiment, the insertion point is fabricated separately from a non-soluble needle shaft. In another embodiment, the insertion point is fabricated in an integrated manner with a substantially soluble needle shaft. Fabrication of a soluble insertion point and/or soluble needle are described in more detail later in conjunction with FIG. 19.

In block 1704, the needle is attached to a needle hub. In an embodiment, the needle hub includes a first portion of a retraction mechanism (e.g., as illustrated in FIG. 7). In another embodiment, the needle hub does not include a retraction mechanism, but instead is designed to provide for full retraction of the needle and insertion point.

In block 1706, the needle is inserted through the catheter connector and catheter tube so that the insertion point extends beyond the distal end of the catheter tube. In another embodiment, the catheter connector may be snapped or otherwise fastened around the needle hub, and the catheter tube may be slid over the needle and attached to the catheter connector.

In block 1708, the end cap is inserted into the blood collection chamber of the needle hub. The catheter device is then sterilized, in block 1710. In an embodiment, sterilization may include irradiating the catheter device (e.g., by gamma or E-beam irradiation) or by exposure to a gaseous sterilization agent (e.g., ethylene oxide gas). These processes may be carried out at controlled temperatures and humidity conditions. After sterilization, the catheter device is ready for packaging. In an embodiment, packaging is performed in a controlled humidity environment, and a desiccant may be included in the package to ensure that moisture does not prematurely degrade the device. The method then ends.

In various embodiments, the catheter connector and needle hub may not be readily slid together during manufacture, due to the various portions of the catheter connector and needle hub. Accordingly, in several embodiments, the needle hub, the catheter connector, or both may be formed from multiple parts, which may be connected together during manufacture of the catheter device.

FIG. 18 illustrates a flowchart of a method for manufacturing a catheter having a substantially soluble insertion needle, in accordance with another embodiment. In the illustrated embodiment, the needle hub includes at least two parts, which are separable, but are designed to be connected during manufacture, as described below. For example, the needle hub may include a shaft part (e.g., part 710, FIG. 7) and a blood collection chamber part (e.g., part 712, FIG. 7).

A method for fabricating a catheter device having such a structure begins, in block 1802, by fabricating an insertion point. In an embodiment, the insertion point is fabricated as a separate device from a soluble or non-soluble needle shaft to which the point may eventually be attached. In another embodiment, the insertion point is fabricated in an integrated manner with a substantially soluble needle shaft. A “soluble needle” means a needle that includes a substantially soluble insertion point and substantially soluble needle shaft. A phrase such as “a needle having a soluble insertion point” is meant to include both a soluble needle and a needle with a non-soluble shaft and a substantially soluble insertion point. Fabrication of a soluble insertion point and/or soluble needle are described in more detail later in conjunction with FIG. 19.

In block 1804, the needle having a soluble insertion point is attached to a first part (e.g., shaft 710, FIG. 7) of the needle hub. In an embodiment, the first part of the needle hub includes a hollow shaft with an exterior diameter that is small enough to slide into the interior channel of the catheter connector. The hollow shaft further includes one or more angled glides, notches, and first glide stop mechanisms.

In block 1806, the first part of the needle hub is inserted into the catheter connector. In an embodiment, the catheter tube is already attached to the catheter connector, and so the needle hub and needle are also inserted through the catheter tube so that the insertion point extends beyond the distal end of the catheter tube. In another embodiment, the catheter tube may be slid over the needle and attached to the catheter connector after the first part of the needle hub is slid into the catheter connector.

In block 1808, a second part (e.g., blood collection chamber part 712, FIG. 7) of the needle hub is attached to the first part of the needle hub. In an embodiment, the second part of the needle hub includes the blood collection chamber, and may have an exterior diameter that is greater than the interior diameter of the catheter connector.

In block 1810, an end cap is inserted into the blood collection chamber of the needle hub. The catheter device is then sterilized, in block 1812, and packaged, and the method ends.

FIG. 19 illustrates a flowchart of a method for fabricating a substantially soluble insertion point or a substantially soluble needle (e.g., blocks 1702, 1802, FIGS. 17, 18), in accordance with an embodiment. The method begins, in block 1902, by preparing a soluble material for further processing, where the soluble material includes one or more soluble substances. The materials selected for the soluble material should result in an end-product with the desired hardness, brittleness, and elasticity that are characteristic for an insertion point or needle. Further, the materials selected should result in an end-product with the desired dissolution characteristics (e.g., fast or slow dissolving).

In an embodiment, the materials selected result in an insertion point that will substantially dissolve when exposed to a dissolution liquid (i.e., a liquid capable of dissolving the soluble material, such as blood or saline, for example) for a period of time less than approximately five minutes. In another embodiment, the materials selected result in an insertion point that will substantially dissolve when exposed to a dissolution liquid for a period of time less than approximately thirty minutes. In still another embodiment, the materials selected result in an insertion point that will substantially dissolve when continuously exposed to a dissolution liquid for a period of time less than approximately twenty-four hours. In still another embodiment, the materials selected result in an insertion point that will substantially dissolve when exposed to a dissolution liquid for a period of time less than approximately seven days.

In an embodiment, the soluble material may have some or all of the following characteristics after processing:

1) the material is soluble within bodily or other fluids (e.g., aqueous fluids);

2) the material is bio-compatible, meaning that the material does not invoke a significant inflammatory or toxic response;

3) the material has mechanical properties that match the application, remaining sufficiently strong until catheter insertion is complete;

4) the material is bio-absorbable, meaning that the material is metabolized in the body after fulfilling its purpose, leaving little or no trace;

5) the material is processable into a final product form (e.g., insertion point and/or needle);

6) the material demonstrates a reasonable shelf life; and

7) the material is readily sterilized.

In an embodiment, the soluble material includes one or more bio-compatible materials, meaning that the material does not invoke a significant inflammatory or toxic response when dissolved in the body. In another embodiment, the soluble material includes one or more bio-compatible and bio-absorbable materials, meaning that the material may is substantially soluble, in vivo, and may be metabolized or further broken down by the body. In other embodiments, soluble insertion point 120 may be formed from one or more other types of materials, which are substantially soluble, but which are not considered to be “bio-compatible” or “bio-absorbable.”

In an embodiment, the soluble material includes one or more materials selected from a group of bio-compatible materials that includes a soluble glass, a soluble bioceramic, a natural biopolymer, a synthetic biopolymer, other bio-compatible materials, other bio-absorbable materials, other soluble materials, or combinations of the like. The term “biopolymer” is used herein to mean a bio-absorbable polymer that disintegrates through biodegradation or bioerosion.

When a soluble bioceramic is selected, the material may include one or more bioceramics selected from a group of materials that includes alumina, calcium phosphate, silica-based glasses, glass ceramics, and pyrolytic carbons. For example, but not by way of limitation, one or more calcium phosphates may be selected from a group of calcium phosphates that includes tetracalcium phosphate, amorphous calcium phosphate, alpha-tricalcium phosphate, beta-tricalcium phosphate, and hydroxyapatite.

When a natural biopolymer is selected, the material may include one or more biopolymers selected from a group of biopolymers that includes a protein and a polysaccharide. When a protein is selected, the material may include one or more proteins selected from a group that includes collagen, keratin, chitosan, fibrinogen, fibronecin, vitronectin, laminin, and gelatin.

When a synthetic biopolymer is selected, the soluble material may include one or more homopolymers or a set of copolymers. Accordingly, a synthetic biopolymer may include one or more polymers (or copolymers) selected from a group of materials that includes esters, polyether-esters, poly(ε-caprolactone), polyanhydrides, polyorthoesters, polyphosphazenes, polyamides, poly(dioxanone), poly(trimethylene carbonate), polyhydroxybutyrate (PHB), polyhydroxyvalerate (PHV), poly(amino acids), polyesteramides, polyesters (e.g., polyglycolide (PGA), polylactide (PLA), poly(dl-lactide) (DLPLA), poly(l-lactide) (LPLA), poly(lactide-co-glycolide) (PGA-PLA), poly(l-lactide-co-glycolide) (PGA-LPLA), poly(dl-lactide-co-glycolide) (PGA-DLPLA), poly(l-lactide-co-dl-lactide) (LPLA-DLPLA), poly(glycolide-co-trimethylene carbonate) (PGA-TMC), poly(glycolice-co-trimethylene carbonate-co-dioxanone) (PDO-PGA-TMC)), modified starches, modified cellulose, polyvinylalcohols, other synthetic bio-absorbable biopolymers, or combinations of the like.

The above examples are not meant to be limiting, and it would be apparent to one of skill in the art, based on the description herein, that a soluble material may be formed from one or more other suitable materials currently in existence or that are developed in the future. In addition, various catalysts, additives, impurities or plasticizers may be included in a soluble material, in various embodiments. For example, but not by way of limitation, one or more medications or other components (e.g., an anti-clotting agent) may be included in a soluble material. Further, a substantially soluble insertion point or a substantially soluble needle may include one or more rigid or flexible reinforcing structures or reinforcing materials within the point or needle.

Referring back to FIG. 19, the soluble material is melt processed at a controlled temperature, for example, using injection molding, compression molding, extrusion, or a similar process, in block 1904. In an embodiment, the result is a structure that has a shape that roughly conforms to the outside shape and topography of the soluble insertion point or needle being formed.

In block 1906, the exterior of the rigid structure is smoothed (e.g., sanded). In block 1908, an interior channel is drilled in the structure, in an embodiment. In an alternate embodiment, the mold could be so designed to inherently produce a rigid structure within which a channel is already formed.

Finally, in block 1910, the insertion point is sharpened to a sharpness that is appropriate for insertion into a body. The method then ends.

The various blocks depicted in FIGS. 11 and 17-19 may be performed in different orders from the orders illustrated and described, while still achieving the same results. Additionally, some blocks may be performed in parallel, rather than sequentially.

Although specific embodiments have been illustrated and described herein, it will be appreciated by those of ordinary skill in the art that any arrangement that is calculated to achieve the same purpose may be substituted for the specific embodiments shown. In particular, retraction mechanisms having different configurations from the specific embodiments described may be used. For example, but not by way of limitation, an alternate retraction mechanism may include complementary threaded members associated with a catheter connector and a needle hub, where retraction is accomplished by twisting the catheter connector with respect to the needle hub.

Adaptations of the invention may be apparent to those of ordinary skill in the art, based on the description herein. Accordingly, this application is intended to cover apparent adaptations or variations of the inventive subject mater.

For example, in embodiments described above, an insertion point is partially retractable to a position inside a distal end of a catheter. In other embodiments, an insertion point may be retractable further into the catheter tube, and/or into the needle hub itself. In still other embodiments, the insertion point and needle may be fully retractable and removable from the catheter. After removal of the soluble insertion point, the needle may be disposed of in a solution that facilitates dissolution of the insertion point. In this manner, the insertion point may eventually be eliminated. Further, in an embodiment, a soluble needle shaft also may eventually be eliminated after it dissolves in a solution. It is manifestly intended that this invention be limited only by the following claims and equivalents thereof.

What is claimed is:

1. A catheter apparatus comprising:
a catheter having a hollow catheter interior, a proximal catheter end, and a distal catheter end; and
a substantially soluble insertion point extending beyond the distal catheter end, wherein the substantially soluble insertion point is formed from one or more substantially soluble materials.

2. The catheter apparatus of claim 1, wherein at least one of the one or more substantially soluble materials includes one or more bio-compatible materials.

3. The catheter apparatus of claim 1, wherein at least one of the one or more substantially soluble materials includes a soluble glass.

4. The catheter apparatus of claim 1, wherein at least one of the one or more substantially soluble materials includes a soluble bioceramic.

5. The catheter apparatus of claim 1, wherein at least one of the one or more substantially soluble materials includes a natural biopolymer.

6. The catheter apparatus of claim 1, wherein at least one of the one or more substantially soluble materials includes a protein.

7. The catheter apparatus of claim 1, wherein at least one of the one or more substantially soluble materials includes a polysaccharide.

8. The catheter apparatus of claim 1, wherein at least one of the one or more substantially soluble materials includes a synthetic biopolymer.

9. The catheter apparatus of claim 1, wherein at least one of the one or more substantially soluble materials is selected from a group of materials that includes a soluble glass, a soluble bioceramic, a natural biopolymer, and a synthetic biopolymer.

10. The catheter apparatus of claim 1, wherein at least one of the one or more substantially soluble materials is selected from a group of materials that includes alumina, calcium phosphate, a silica-based glass, a glass ceramic, and a pyrolytic carbon.

11. The catheter apparatus of claim 1, wherein at least one of the one or more substantially soluble materials is selected from a group of materials that includes an ester, a polyether-ester, a poly(ε-caprolactone), a polyanhydride, a polyorthoester, a polyphosphazene, a polyamide, a poly(dioxanone), a poly(trimethylene carbonate), a polyhydroxybutyrate (PHB), a polyhydroxyvalerate (PHV), a poly(amino acid), a polyesteramide, and a polyester.

12. The catheter apparatus of claim 1 wherein at least one of the one or more substantially soluble materials is selected from a group of materials that includes polyglycolide (PGA), polylactide (PLA), poly(dl-lactide) (DLPLA), poly(l-lactide) (LPLA), poly(lactide-co-glycolide) (PGA-PLA), poly(l-lactide-co-glycolide) (PGA-LPLA), poly(dl-lactide-co-glycolide) (PGA-DLPLA), poly(l-lactide-co-dl-lactide) (LPLA-DLPLA), poly(glycolide-co-trimethylene carbonate) (PGA-TMC), poly(glycolice-co-trimethylene carbonate-co-dioxanone) (PDO-PGA-TMC), modified starches, modified cellulose, and polyvinylalcohols.

13. The catheter apparatus of claim 1, further comprising:
a needle shaft coupled to the insertion point at a first end of the needle shaft; and
a needle hub coupled to a second end of the needle shaft.

14. The catheter apparatus of claim 13, wherein the needle shaft is formed from a substantially soluble material that includes one or more bio-compatible materials.

15. The catheter apparatus of claim 13, further comprising:
a catheter hub coupled to the catheter at the proximal catheter end; and
a retraction mechanism to enable retraction of the insertion point into the hollow catheter interior.

16. The catheter apparatus of claim 15, wherein the retraction mechanism comprises:
a first portion of the retraction mechanism attached to the needle hub; and
a second portion of the retraction mechanism attached to the catheter connector.

17. The catheter apparatus of claim 13, wherein the needle hub comprises:
a connector portion to attach the catheter apparatus to fluid-delivery tubing.

18. A catheter apparatus comprising:
a catheter having a hollow catheter interior, a proximal catheter end, and a distal catheter end; and
a substantially soluble needle, to fit within the hollow catheter interior, and which includes a hollow needle channel, a proximal needle end, a distal needle end, and an insertion point proximate to the distal needle end, and wherein the substantially soluble needle is formed from one or more substantially soluble materials.

19. The catheter apparatus of claim 18, wherein the insertion point extends beyond the distal catheter end.

20. The catheter apparatus of claim 18, further comprising:
a needle hub coupled to the proximal needle end;
a catheter hub coupled to the catheter at the proximal catheter end; and
a retraction mechanism to enable retraction of the insertion point into the hollow catheter interior.

21. The catheter apparatus of claim 20, wherein the retraction mechanism comprises:
a first portion of the retraction mechanism attached to the needle hub; and
a second portion of the retraction mechanism attached to the catheter connector.

22. The catheter apparatus of claim 18, wherein at least one of the one or more substantially soluble materials includes one or more bio-compatible materials.

23. The catheter apparatus of claim 18, wherein at least one of the one or more substantially soluble materials is selected from a group of materials that includes a soluble glass, a soluble bioceramic, a natural biopolymer, and a synthetic biopolymer.

24. A method for using an apparatus that includes a catheter, the method comprising:
inserting a substantially soluble insertion point of the apparatus into a body,
wherein the substantially soluble insertion point extends beyond a distal end of the catheter.

25. The method of claim 24, further comprising:
partially retracting the substantially soluble insertion point into the distal end of the catheter.

26. The method of claim 24, further comprising:
completely removing the substantially soluble insertion point from the apparatus; and
disposing of the substantially soluble insertion point in a solution to dissolve the substantially soluble insertion point.

27. A method for making a catheter apparatus, the method comprising:
forming a substantially soluble insertion point;
attaching a proximal end of a needle to a needle hub, wherein a distal end of the needle is connected to the substantially soluble insertion point; and
inserting the needle into a catheter.

28. The method of claim 27, wherein forming the substantially soluble insertion point comprises:
forming the substantially soluble insertion point from one or more substantially soluble materials selected from a group of materials that includes a soluble glass, a soluble bioceramic, a natural biopolymer, and a synthetic biopolymer.

29. The method of claim 27, wherein inserting the needle comprises:
inserting the needle so that the substantially soluble insertion point extends beyond a distal end of the catheter.
